(19) United States
(12) Patent Application Publication
(10) Pub. No.: US 2021/0346559 A1
(43) Pub. Date: Nov. 11, 2021

(54) Purification and Sanitization Using Directed Energy

(71) Applicant: Argosy Holdings, LLC, Jenkintown, PA (US)

(72) Inventors: Michael Weiner, Jupiter, FL (US); Maxwell M. Newman, Tampa, FL (US); Michael Richard Manning, Oxford, PA (US); Aaron B. Cohen, Penn Valley, PA (US)

(21) Appl. No.: 17/313,351

(22) Filed: May 6, 2021

Related U.S. Application Data
(60) Provisional application No. 63/021,310, filed on May 7, 2020.

(51) Int. Cl. A61L 2/24; A61L 2/26; A61L 2/08; A61L 2/03; A61L 9/015; A61L 9/18

(52) U.S. Cl. CPC A61L 2/24; A61L 2/26; A61L 2/08; A61L 2/03; A61L 9/015; A61L 2209/212; A61L 2202/11; A61L 2202/14; A61L 2202/16; A61L 2202/17; A61L 2209/11; A61L 9/18

(57) ABSTRACT

Systems and methods using EMR and other directed energies emitted at surfaces, objects and body parts, to kill, neutralize, eliminate, remove or otherwise treat or influence objects, species, dirt, weeds, worms, pests, bacteria, viruses, parasites, fungi, other pathogens and other organic and inorganic matter from target surfaces are disclosed. One embodiment includes selecting an at least one first set of parameters of directed energy, wherein the at least one first set of parameters may include one or more of a wavelength, frequency, amplitude, duration, acceleration, deceleration, pulsation, and pulsation period; emitting the directed energy based on the selected at least one first set of parameters at a target surface; measuring in real-time the temperature of the target surface; and pausing the emitting of directed energy once a trigger occurs. This process may be repeated with different sets of parameters and directed energies and carried out in series or simultaneously.

### CROSS-REFERENCES TO RELATED APPLICATIONS

This application claims the benefit and priority of U.S. Provisional Application Ser. No. 63/021,310, filed on May 7, 2020, which is hereby incorporated by reference herein in its entirety, including all references and appendices cited therein, for all purposes, as if fully set forth herein.

### FIELD OF INVENTION

The present technology pertains to systems and methods for the utilization of electromagnetic radiation and/or other directed energies in techniques and technologies including, but not intended to be limited to, for sterilization, purification, sanitation, disinfection and biotherapeutic uses.

### SUMMARY

In various embodiments the present technology is directed to a method for treating surfaces, comprising selecting an at least one first set of parameters of directed energy, wherein the at least one first set of parameters may include one or more of a wavelength, frequency, amplitude, duration, acceleration, deceleration, pulsation, and pulsation period; emitting the directed energy based on the selected at least one first set of parameters at a target surface; measuring in real-time the temperature of the target surface; and pausing the emitting of directed energy once a trigger occurs, wherein the trigger may be one or more of a passage of a prescribed length of time, a reaching of a threshold temperature, and a maintenance of a temperature for a prescribed length of time.

In several embodiments, this method may further comprise selecting an at least one second set of parameters of directed energy, wherein the at least one second set of parameters may include one or more of a wavelength, frequency, amplitude, duration, acceleration, deceleration, pulsation, and pulsation period; emitting the directed energy based on the selected at least one second set of parameters at the target surface; and pausing the emitting of directed energy once a trigger occurs, wherein the trigger may be one or more of a passage of a prescribed length of time, a reaching of a threshold temperature, and a maintenance of a temperature for a prescribed length of time. The method may further comprise selecting an at least one third set of parameters of directed energy, wherein the at least one second set of parameters may include one or more of a wavelength, frequency, amplitude, duration, acceleration, deceleration, pulsation, and pulsation period; emitting the directed energy based on the selected at least one third set of parameters at the target surface, wherein the at least one third set of parameters may include one or more of a wavelength, frequency, amplitude, duration, acceleration, deceleration, pulsation, and pulsation period; and pausing the emitting of directed energy once a trigger occurs, wherein the trigger may be one or more of a passage of a prescribed length of time, a reaching of a threshold temperature, and a maintenance of a prescribed temperature for a prescribed length of time.

### BRIEF DESCRIPTION OF THE DRAWINGS

In the description, for purposes of explanation and not limitation, specific details are set forth, such as particular embodiments, procedures, techniques, etc. to provide a thorough understanding of the present technology. However, it will be apparent to one skilled in the art that the present technology may be practiced in other embodiments that depart from these specific details.

The accompanying drawings, where like reference numerals refer to identical or functionally similar elements throughout the separate views, together with the detailed description below, are incorporated in and form part of the specification, and serve to further illustrate embodiments of concepts that include the claimed disclosure and explain various principles and advantages of those embodiments.

The methods and systems disclosed herein have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

FIG. 1 presents an embodiment of a carpet, floor or surface treatment device.

FIG. 2 presents an embodiment of a system for air vent treatment.

FIG. 3 presents an embodiment of a hand and object applied-air treatment system.

FIG. 4 presents another embodiment of a hand and object applied-air treatment system.

FIG. 5 presents a system for treating multiple objects by placing them through a treatment machine.

FIG. 6 presents a table of various wavelengths, frequencies and amounts of electromagnetic radiation and other directed energies utilized by the systems and methods herein to achieve the discussed outcomes.

FIG. 7 presents a flow chart for one embodiment of a method to disinfect, sanitize, clean or otherwise treat a target surface, object or body part.

### DETAILED DESCRIPTION

The approaches described in this section could be pursued but are not necessarily approaches that have previously been conceived or pursued. Therefore, unless otherwise indicated, it should not be assumed that any of the approaches described in this section qualify as prior art merely by virtue of their inclusion.

The Coronavirus/Covid-19 has exposed deep flaws in the current sanitization, purification and therapeutic structures and systems deployed in our shared spaces, homes, workplaces and broader communities. To arrest the spread of disease during this pandemic, people have resorted to wiping down every object and surface, washing hands more frequently, disinfecting air and installing expensive air purification systems, and wearing masks widely. The costs, time and effort spent on installing and maintaining all these systems as well as undertaking constant cleaning, sanitization and health and safety promotion cannot be sustainable from either a human or financial resource perspective.

Furthermore, during this pandemic, a host of chemicals, chemical agents and biologically toxic methods have been utilized to disinfect or otherwise treat a variety of surfaces, spaces, clothes and body parts. These chemical agents pose a variety of short and long-term health problems to the individual as well as to the environment. Alcoholic hand disinfectants for example damage the layers of the skin and overuse can lead to poisoning, while chemical agents sprayed in the air can be harmful to human, animal and plant life, may damage the skin or may inflame the lungs. A host of other problems from the use and overuse of these agents have been recorded and are known in the art.

The technologies presented in this patent application are intended as safer alternative systems and methods for beneficially treating surfaces, objects, body parts, contained spaces, open spaces, biological systems and environments (collectively referred to in this document as “targets”) while avoiding the deployment of chemical and biologically toxic agents. Presented herein are systems and methods for the sanitization, purification and/or other beneficial treatment of surfaces such as floors, carpets, tables and desks. Also presented are systems and methods directed to air vent sanitization, purification and/or other beneficial treatment. Also presented are systems and methods for the sanitization, purification and/or other beneficial treatment of objects as well as body parts. These alternative solutions not only provide chemical agent-free systems and methods for sanitization and purification but can also provide or improve healing or other biologically therapeutic results to humans, animals and the broader environment.

The technologies discussed herein utilize waves of the electromagnetic radiation (EMR) spectrum as well as other waves such as acoustic waves (collectively referred to in this document as “directed energy” or “directed energies”) to kill, neutralize, eliminate, remove and/or otherwise influence objects, species, dirt, weeds, worms, pests, bacteria, viruses, parasites, fungi, other pathogens and other organic and inorganic matter (collectively referred to in this document as “antagonists”). The technologies presented accomplish this by exposing antagonists and/or environments containing or suspected of containing antagonists to directed energies at specific wavelengths, frequencies and/or other selectable controlling directed energy parameters. These technologies produce and emit directed energies via a number of sources including and not limited to any number and combination of: light emitting diodes (LEDs), lights, lasers, laser diodes, pulsed electromagnetic field emitters and waveguides, cavity magnetrons and other magnetic stimulation devices, radio frequency oscillators, etc. (collectively referred to in this document as “directed energy sources”).

In addition to the specific uses of the systems and methods presented, the technologies discussed in this document may also be applied and used to decontaminate and treat water supplies, move or remove organic and inorganic compounds from various environmental systems and media, decontaminate and promote a healthy soil environment, promote biological life and health including in insects, bee colonies, algae, sea plants and other beneficial organisms, eliminate dirty energy waves and increase the balance of biologically safe and beneficial energy waves in the environment, and enhance or increase the efficiency of natural immunity and healing mechanisms in humans and animals. The promotion of life, health, propagation, etc. in living organisms under treatment with the systems and devices presented in this document may or may not require manipulation of different parameters of directed energies than those prescribed for the killing, elimination or other influence of antagonists.

In various embodiments, the present technology is directed to selecting a first wavelength, frequency or other controlling parameter of electromagnetic radiation and/or other directed energy or energies for treating a target; emitting the selected wavelength, frequency or other controlling parameter of directed energy/energies at, on, towards or near the target; measuring in real-time the surface or internal temperature of the target; and pausing the emitting of directed energy/energies once a trigger occurs—where a trigger is defined as the passage of a prescribed length of time, the reaching of a threshold temperature, or the maintenance of a prescribed temperature for a prescribed length of time. In various embodiments this process may be repeated simultaneously or serially with second and perhaps third selected wavelengths, frequencies or other controlling parameters of other directed energies for more robust treatment of the target.

### Surface Treatment Device

In various embodiments, systems and methods to sanitize, purify, clean and/or otherwise beneficially treat ground surfaces such as floors, carpets, lawns and other paved or covered ground areas are presented herein (collectively referred to in this document as “ground surface treatment device”). In various embodiments, systems and methods to sanitize, purify, clean and/or otherwise treat surfaces extant within spaces and areas such as on furniture, machinery, equipment, etc. are presented herein (collectively referred to in this document as “stationary surface treatment device”). In several embodiments, “surface” may refer to surfaces of any object or body part, and devices and machines for treating surfaces of objects or body parts may also make use of the systems and methods disclosed herein, and could do so in various forms, including handheld devices, miniaturized devices, or larger full body size devices, chambers, rooms or machines (these devices, the ground surface treatment devices, and the stationary surface treatment devices are collectively referred to in this document as “surface treatment devices”). In various embodiments, robotic or hand-held surface treatment devices, employing directed energy sources to be directed at targets in order to kill, eliminate and/or otherwise influence antagonists are presented herein. In several embodiments, the surface treatment device may be powered by battery or a power outlet. In various embodiments, the surface treatment device is capable of being remotely controlled, can be controlled by a user, or runs and directs itself by detecting areas it has not treated, or in which it detects antagonists. In various embodiments, the surface treatment device may be manually carried, moved, pushed or pulled into and onto different areas and target surfaces. In various embodiments, the surface treatment device contains tires, wheels and/or rotatable discs to allow it to roll, move or be moved.

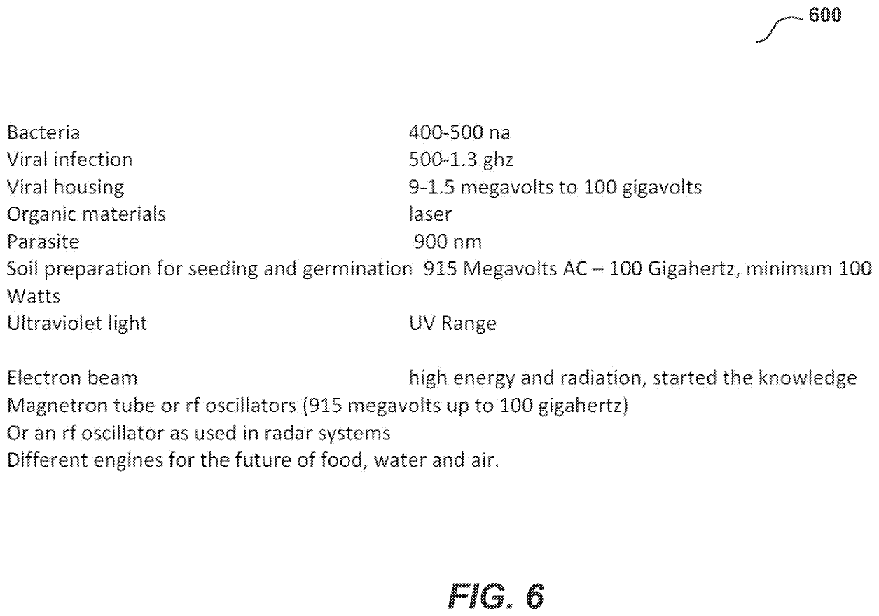

In several embodiments, the surface treatment device may be deployed in a modifiable and foldable form, which may contain larger tires, wheels and/or rotatable discs. In various embodiments, the surface treatment device can be purpose-built or can be switched from being used in indoor environments such as a home, office, or institutional building to being used outdoors or outside of a home, office or institutional building. In various embodiments, the ground surface treatment device may be used on lawns or outdoor areas as an alternative or complementary treatment to poisons, weed killers, pesticides and/or fertilizers. In various embodiments of the surface treatment device, specific wavelengths, frequencies and/or other controlling parameters of directed energies may be selected depending on the functionality desired or the surface or space (target) being treated. (See FIG. 6. herein.) In various embodiments, a closed loop temperature sensor may be included in or used with the surface treatment device to monitor the temperature of the surface being treated. In several embodiments, a timer unit may be included in or used with the surface treatment device in order to measure certain temporal parameters such as the time the device has been active, the length of time the current settings or treatment parameters have been used, the length of time a specific surface has been treated by specific directed energies and/or the length of time a surface being treated has been at a certain temperature (collectively, the “triggers”). In various embodiments, the surface treatment device may beep, indicate, signal or produce an animation, message or alert to the user, or in various embodiments may automatically stop or alter the treatment process once a trigger occurs, for example a prescribed threshold surface temperature is reached, or a prescribed surface temperature has been maintained for a prescribed length of time.

In various embodiments, once the surface treatment device is situated at an area or surface to be treated (a target), whether it was moved there or moved itself there, the initiation of treatment may be affected by an operator of the device or automatically. In various embodiments, the wavelengths, frequencies and/or other controlling parameters of directed energies as shown in FIG. 6. hereto may be employed by the surface treatment device to produce desired outcomes. In various embodiments, a combination of directed energies and/or a combination of wavelengths, frequencies and/or other controlling parameters of a specific directed energy may be emitted simultaneously or serially in order to treat a target, with one range of directed energies or wavelengths/frequencies/parameters being emitted first for a prescribed period of time followed by another range of directed energies or wavelengths/frequencies/parameters being emitted for a second prescribed period of time, perhaps followed by further phases if necessary. In several embodiments, each phase of directed energy emission can have a prescribed duration in this manner, or their respective durations may be based on a threshold temperature created on, below or inside a target by each respective phase of treatment or on a temperature created on, below or inside a target by each respective phase of treatment that is maintained over a prescribed length of time. For example, in various embodiments the target's surface or internals may need to reach 220° F. to produce the desired result while avoiding burning or scorching, and in various other embodiments the target's surface or internals may need to be heated to 220° F. and maintained at that temperature for 1-10 seconds or more to reach the desired result while avoiding burning or scorching. In other embodiments, temperatures lower or higher than 220° F. and durations of temperature maintenance above or below ten seconds may be prescribed. In various embodiments, once the target surface area is determined to have been successfully treated, then the surface treatment device may be programmed to move automatically or may be manually controlled to move on to another surface area or target in the field of treatment.

### Air Vent Treatment System

In various embodiments, systems and methods to sanitize, purify, clean and/or otherwise beneficially treat air circulation and ventilation systems and the air they carry and transport are presented herein (collectively referred to as “air ventilation treatment systems”). In various embodiments, air ventilation treatment systems can be mobile or movable for use in airplanes and other transportation means, hospitals, schools and other institutional buildings, industrial settings, medical or scientific laboratories, manufacturing facilities, etc. or may be temporarily or permanently installed in such places. In various embodiments, air ventilation treatment systems allow the ingress of air into a vent and capture and/or remove antagonists from the air with an electrostatic grid. The electrostatic grid may be installed at the initial point where air enters a vent or in some embodiments adjacent to the vent entry point. In various embodiments, the electrostatic grid runs across a large portion of, or alternatively throughout, an entire air vent. In various embodiments, the air passing through a vent and treated electrostatically is then exposed to directed energy sources which may be placed on all sides of the vent, on some sides or on any one or more sides; in various embodiments, such directed energy sources may line an entire air vent and the entire area through which air will travel. In various embodiments, ozone may be added through a separate input mechanism, inlet, duct or by other methods to further treat the air and neutralize organic matter.

In various embodiments, a closed loop temperature sensor may be included in or used with the air ventilation treatment system to monitor the temperature of air moving through a vent or of the vent itself. In various embodiments, a timer unit may be included in or used with the air ventilation treatment system in order to measure certain temporal parameters such as the time the device has been active, the length of time the current settings or treatment parameters have been used, the length of time a specific vent has been treated by specific directed energies and/or the length of time a vent or volume of air being treated has been at a certain temperature (collectively, the “triggers”). In various embodiments, the air ventilation treatment system may beep, indicate, signal or produce an animation, message or alert to the user or individual controller, or in various embodiments may automatically stop or alter the treatment process upon the occurrence of a trigger including a prescribed threshold air and/or vent temperature is reached or a prescribed air and/or vent temperature has been maintained for a prescribed length of time.

In various embodiments, the directed energy sources may be configured to cause air moving through a vent and/or the vent itself to reach a prescribed threshold temperature or to reach and maintain a certain prescribed temperature for a prescribed length of time. In various embodiments, once a body or mass of air or a vent itself has met a threshold temperature, or a temperature for a certain length of time has been maintained, then the air may be allowed to flow out of the vent into the rest of the system, the room or other space and is replaced by ‘new’ untreated, air. In various embodiments, the wavelengths, frequencies and/or other controlling parameters of directed energies as shown in FIG. 6. hereto may be employed by the air ventilation treatment system to produce desired outcomes. In many embodiments, a combination of directed energies and/or a combination of wavelengths, frequencies and/or other controlling parameters of a specific directed energy may be emitted simultaneously or serially in order to treat a vent or a volume of air, with one range of directed energies or wavelengths/frequencies/parameters being emitted first for a prescribed period of time followed by another range of directed energies or wavelengths/frequencies/parameters being emitted for a second prescribed period of time, perhaps followed by further phases if necessary. Each phase of directed energy emission can have a prescribed duration in this manner, or their respective durations may be based on a threshold temperature created in the air or on the vent by each respective phase of treatment or on a temperature created in the air or on the vent by each respective phase of treatment that is maintained over a prescribed length of time. In various embodiments, air flow actuators are included in the air ventilation treatment system to facilitate the movement of air in and out of the system.

### Hand/Object Treatment System

In various embodiments, systems and methods to sanitize, purify, clean and/or otherwise beneficially treat objects, hands and/or other parts of the human body are presented herein (collectively referred to in this document as “object and body treatment device”). In various embodiments, presented are systems and methods which may include one or more forced air devices similar to a hand drying machine or other air application system, the object and body treatment device containing a number of directed energy sources placed in or around the parts of the device that apply air to a target. In various embodiments, the directed energy sources are directed towards the areas where objects and/or body parts to be treated are placed. In some embodiments, the directed energy sources are directed towards the passage of air. In various embodiments, a number of placements or configurations of directed energy sources are possible both on the interior and exterior of the air application or object and body treatment device. In various embodiments, drying of the target may or may not also be intended by the device or may be incidental to the operation of the device.

In various embodiments, an object or body part is placed near, in front of, inside or at an alternative placement relative to the object and body treatment device whereby it is sensed by one or more sensors that activate either the air application mechanism, expelling air onto the hand(s) or other object(s) placed near it, or activating the directed energy sources to emit their respective radiation at the object being treated (target), or both. In various embodiments, both air application and the emitting of one or more forms of directed energy are done simultaneously, while in various embodiments one may start and end before another begins, or another starts and then each one ends depending on certain parameters or programmed settings to achieve the desired outcomes.

In various embodiments, a temperature sensor, a timer, or both are included in the object and body treatment system. The temperature sensor may be placed to detect the surface and/or internal temperature of the target at which the directed energy or energies are is/are emitted. The temperature sensor may be pre-programmed to detect certain temperature thresholds, certain temperatures, or certain temperatures for certain lengths of time. Once the temperature sensor detects the target reaching certain temperatures, or in some embodiments certain temperatures for certain lengths of time, it may either cause signaling component(s) of the system to beep, alert or notify the user in some way, or cause the deactivation of the directed energy source(s), the air application, or both. A timer may also be included in the system and work simultaneously with or separately from the temperature sensor. The timer may be programmed to function in various ways, including to turn on as soon as directed energy is emitted, when the target surface or internal temperature reaches a prescribed threshold, and/or when a prescribed temperature is maintained for a prescribed period of time. The timer may be connected to a signaling device such a beeper, an alarm, bell, light, diode or other sound or light producing component to indicate the beginning and/or end of the process. In various embodiments, the timer may be programmed to turn off the air application component, the directed energy source(s), or both upon reaching a prescribed time length.

In various embodiments, the wavelengths, frequencies and/or other controlling parameters of directed energies as shown in FIG. 6. hereto may be employed by the object and body treatment device to produce desired outcomes. In various embodiments, a combination of directed energies and/or a combination of wavelengths, frequencies and/or other controlling parameters of a specific directed energy may be emitted simultaneously or serially in order to treat a target, with one range of directed energies or wavelengths/frequencies/parameters being emitted first for a prescribed period of time followed by another range of directed energies or wavelengths/frequencies/parameters being emitted for a second prescribed period of time, perhaps followed by further phases if necessary. Each phase of directed energy emission can have a prescribed duration in this manner, or their respective durations may be based on a threshold temperature created on or inside a target by each respective phase of treatment or on a temperature created on or inside a target by each respective phase of treatment that is maintained over a prescribed length of time. These different directed energy phases, wavelengths, frequencies, etc. may also be emitted simultaneously by the same or different directed energy sources.

### Conveyor Belt Object Treatment System

In various embodiments, systems and methods to sanitize, purify, clean and/or otherwise beneficially treat objects that are moved through a machine or device via a transport mechanism are presented herein (collectively referred to in this document as “conveyor belt object treatment system”). In various embodiments, an open or closed system that utilizes a conveyor or transport belt is presented, some embodiments of this system being intended to include one or more places of entry and one or more places of exit for targets. As a target is placed into the system, it is moved via the conveyor or transport belt and is automatically treated and transported to the exit where treated objects are placed. In various embodiments, directed energy source(s) is/are situated above the targets being treated; in various embodiments, these sources are placed in different configurations including and not limited to surrounding the target on one side, two sides or all sides. In exemplary embodiments, the transport conveyor belt may move at a speed of 1 foot per second. In various embodiments, a temperature sensor(s) for determining the temperature(s) inside the system and/or the target are included or employed. Various embodiments also employ timers and other time keeping mechanisms. As target(s) are moved through the system via the belt, the temperature created on or in the target by the directed energy or energies may be maintained at a certain threshold and/or a certain length of time, for example at 200-220° F. for 1-10 seconds, to produce the desired result while avoiding burning or scorching the target(s). In various embodiments, the wavelengths, frequencies and/or other controlling parameters of directed energies as shown in FIG. 6. hereto may be employed by the conveyor belt object treatment system to produce desired outcomes. In various embodiments, a combination of directed energies and/or a combination of wavelengths, frequencies and/or other controlling parameters of a specific directed energy may be emitted simultaneously or serially in order to treat a target, with one range of directed energies or wavelengths/frequencies/parameters being emitted first for a prescribed period of time followed by another range of directed energies or wavelengths/frequencies/parameters being emitted for a second prescribed period of time, perhaps followed by further phases if necessary. Each phase of directed energy emission can have a prescribed duration in this manner, or their respective durations may be based on a threshold temperature created on or inside a target by each respective phase of treatment or on a temperature created on or inside a target by each respective phase of treatment that is maintained over a prescribed length of time. These different directed energy phases, wavelengths, frequencies, etc. may also be emitted simultaneously by the same or different directed energy sources. Many objects can be sanitized including paper bills, plastics, electronics, food items, etc. Thus, for example, if the target was a stack of cash bills, the system would detect the temperature of the bills in the middle of the stack and inform the progress of the treatment accordingly.

In most embodiments, once an object has been treated, the emission of directed energy may be halted, or the temperature of the closed system dropped, or the conveyor belt increased in speed to move the treated target to exit the system.

While the present technology is capable of embodiment in many different forms, there is shown in the drawings and will herein be described in detail several specific embodiments with the understanding that the present disclosure is to be considered as an exemplification of the principles of the present technology and is not intended to limit the technology to the embodiments illustrated.

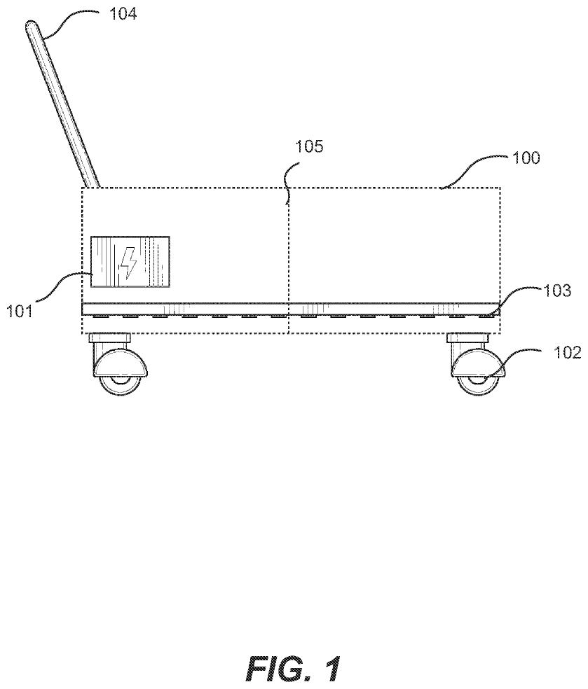

FIG. 1 presents an embodiment of a carpet, floor, lawn, or surface sanitization device 100. The device 100 may include a battery or power source 101, wheels, tires, discs, or other rotating components 102, directed energy source(s) 103 directed towards the surface to be treated. The device may also have a handle 104 to allow a user to move or maneuver the device. The device may also be foldable across fold 105 to allow easy carry and storage.

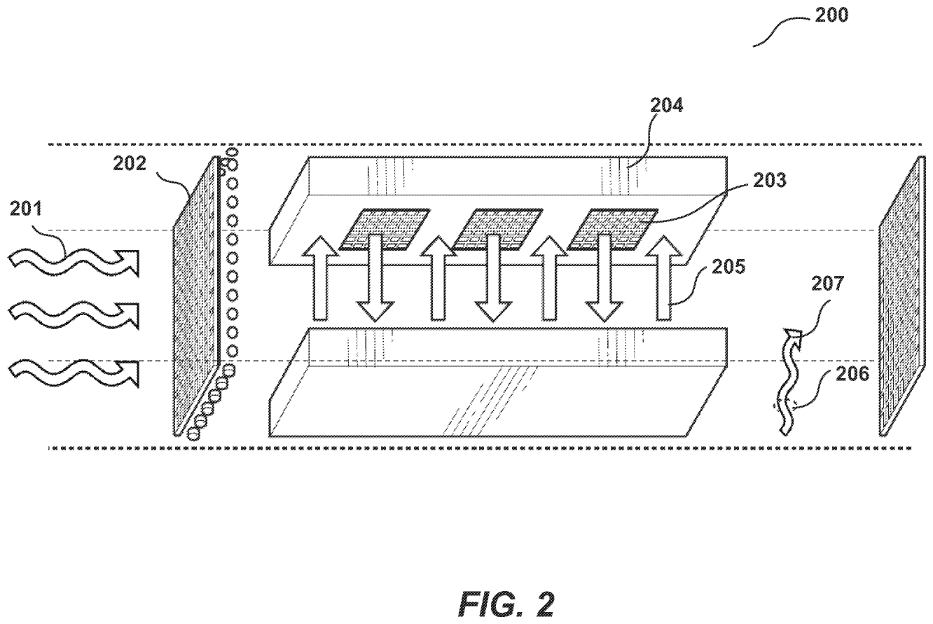

FIG. 2 presents one embodiment of an air vent purification system 200. Shown is mass of air 201 upon entering the vent/chamber, going through electrostatic grid 202 which removes or isolates particles in the air, and then going through directed energy source(s) 203 placed as a bar, in or on a bar or in a bar formation 204. These directed energy source(s) 203 may be placed on one or more or all sides of the vent to produce directed energy waves (directed energies) 205 into the mass of air 201. In some embodiments an ozone inlet vent 206 is included to add ozone 207 to the air that has been treated.

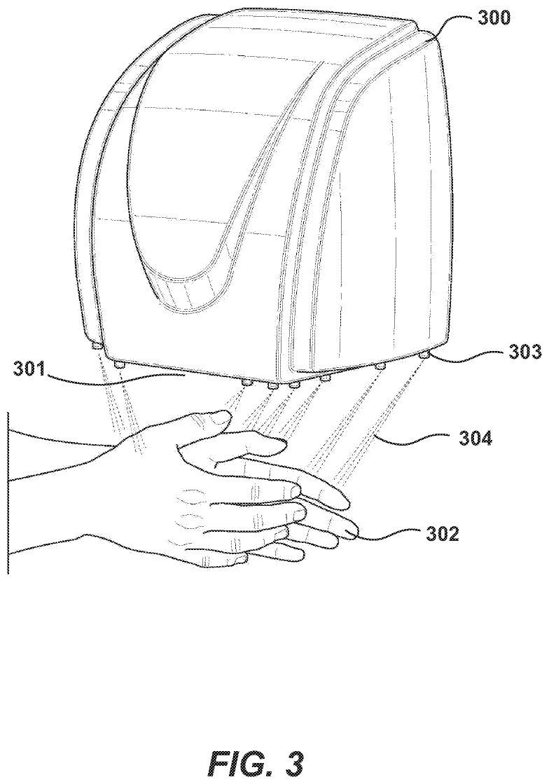

FIG. 3 presents an embodiment of an object and body part applied-air treatment device 300. Presented is an air outlet 301 for treating and possibly drying hands or objects 302, one or more directed energy source(s) 303 that produce directed energy waves (directed energies).

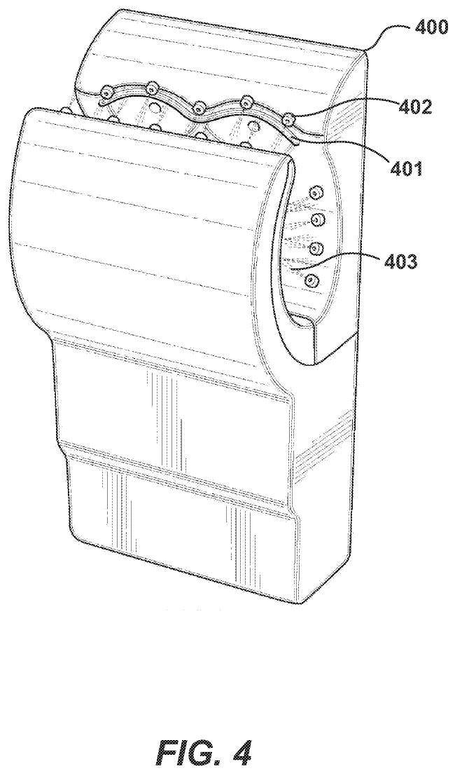

FIG. 4 presents another embodiment of an object and body part applied-air treatment device. Presented is an air outlet 401 for treating and possibly drying hands or objects, one or more directed energy source(s) 402 that produce directed energy waves (directed energies) 403.

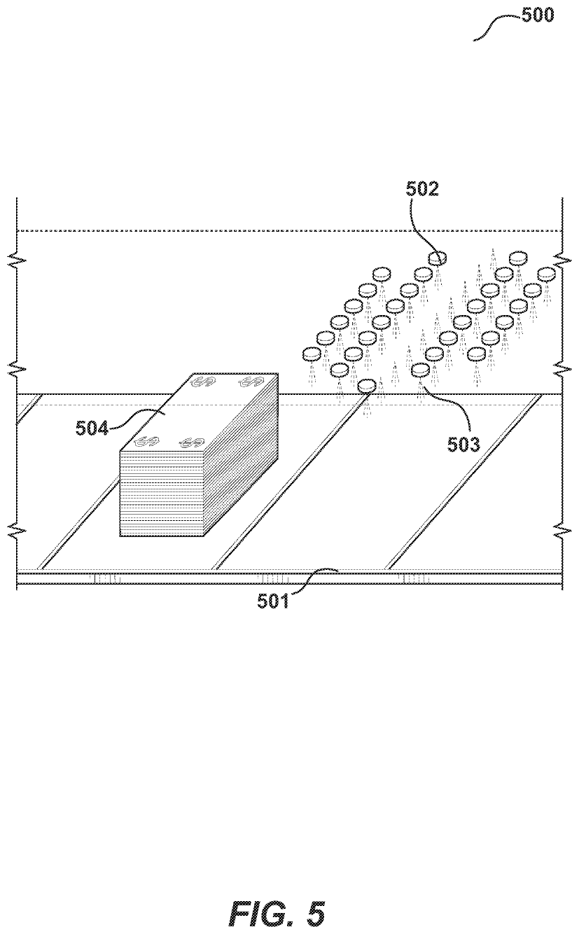

FIG. 5 presents a system for treating one or more targets by placing them through a treatment machine 500. The machine 500 includes a conveyor belt 503 which transports one or more objects 504. As the machine moves the one or more targets 503, it emits directed energy from one or more directed energy source(s) 502 to treat the one or more targets 504 with directed energy waves (directed energies). The machine 500 may also include other components, including a temperature sensor or timer device.

FIG. 6 presents a table 600 of parameters including different wavelengths, frequencies and amounts of energy utilized by the systems and methods described in this document to achieve different outcomes including but not limited to killing, eliminating, neutralizing, removing or otherwise influencing certain antagonists. For example, bacteria may be eliminated or killed by using a wavelength of EMR in the range of 400-500 nanometers. Viruses may be eliminated by using an energy frequency range between 500 MHz to 1.3 GHz. Parasites may be removed or killed by an EMR wavelength of 900 nanometers. Other antagonists may or may not require different wavelengths, frequencies and/or amounts of energy to be treated. Other parameters of directed energy application such as amplitude, duration, acceleration and deceleration, pulsation and pulsation period, etc. may be employed in the systems described in this document in order to effect desired outcomes.

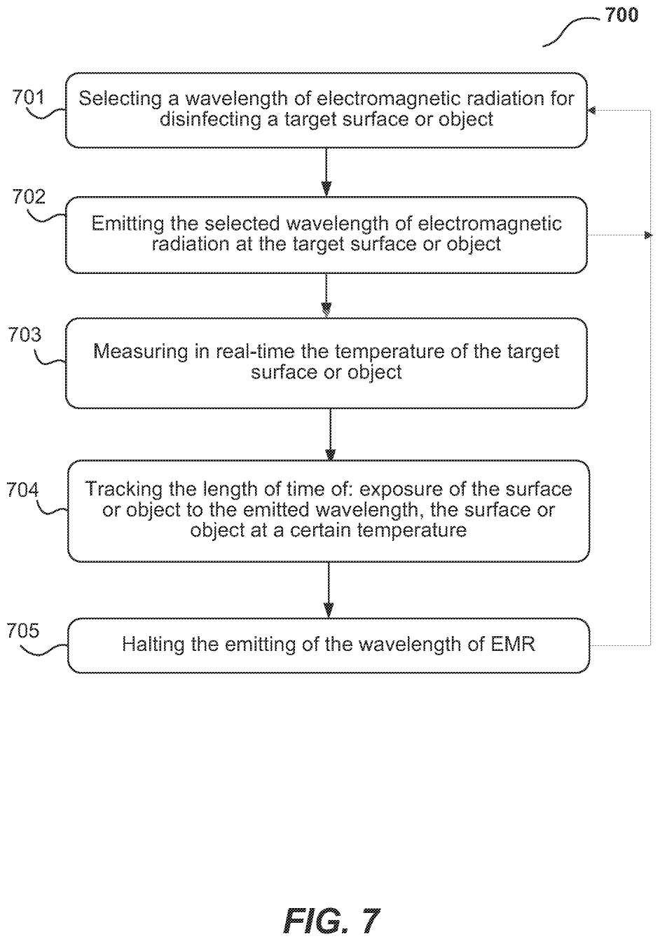

FIG. 7 presents a flow chart for one embodiment of a method 700 to treat a target. A wavelength and/or other set of parameters of a directed energy or energies is/are selected 701 for treating the target. The energy at that wavelength and/or other set of parameters is emitted 702 at or towards the target. The temperature of the target is measured 703. The length of time the wavelength and/or other set of energy parameters is emitted at or towards the target for, or the length of time that a temperature or a temperature range is maintained, is tracked 704. Finally, once a prescribed length of time has passed, or a prescribed temperature has been reached, or a temperature maintained for a prescribed period of time, the emission of the directed energy or energies at that wavelength and/or other set of parameters is halted or paused 705. In some embodiments, once a wavelength and/or other set of energy parameters is emitted, another wavelength and/or other set of energy parameters is then selected and emitted at the same time to address a specified antagonist. In some embodiments, once the emission of the first directed energy wavelength or other set of parameters has been halted or paused, another directed energy, wavelength of the first selected directed energy and/or other set of energy parameters is selected to target different antagonists and the process repeats itself.

The foregoing detailed description includes references to the accompanying drawings, which form a part of the detailed description. The drawings show illustrations in accordance with exemplary embodiments. These example embodiments, which are also referred to herein as “examples,” are described in enough detail to enable those skilled in the art to practice the present subject matter.

The embodiments can be combined, other embodiments can be utilized, or structural, logical, and electrical changes can be made without departing from the scope of what is claimed. The following detailed description is, therefore, not to be taken in a limiting sense, and the scope is defined by the appended claims and their equivalents. In this document, the terms “a” or “an” are used, as is common in patent documents, to include one or more than one. In this document, the term “or” is used to refer to a nonexclusive “or,” such that “A or B” includes “A but not B,” “B but not A,” and “A and B,” unless otherwise indicated. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

The corresponding structures, materials, acts, and equivalents of all means or step plus function elements in the claims below are intended to include any structure, material, or act for performing the function in combination with other claimed elements as specifically claimed. The description of the present technology has been presented for purposes of illustration and description and is not intended to be exhaustive or limited to the invention in the form disclosed. Many modifications and variations will be apparent to those of ordinary skill in the art without departing from the scope and spirit of the invention. Exemplary embodiments were chosen and described to best explain the principles of the present technology and its practical application, and to enable others of ordinary skill in the art to understand the invention for various embodiments with various modifications as are suited to the particular use contemplated.

While specific embodiments of, and examples for, the system are described above for illustrative purposes, various equivalent modifications are possible within the scope of the system, as those skilled in the relevant art will recognize. For example, while processes or steps are presented in a given order, alternative embodiments may perform routines having steps in a different order, and some processes or steps may be deleted, moved, added, subdivided, combined, and/or modified to provide alternative or sub-combinations. Each of these processes or steps may be implemented in a variety of different ways. Also, while processes or steps are at times shown as being performed in series, these processes or steps may instead be performed in parallel or may be performed at different times.

The various embodiments described above are presented as examples only, and not as a limitation. The descriptions are not intended to limit the scope of the present technology to the forms set forth herein. To the contrary, the present descriptions are intended to cover such alternatives, modifications, and equivalents as may be included within the spirit and scope of the present technology as appreciated by one of ordinary skill in the art. Thus, the breadth and scope of a preferred embodiment should not be limited by any of the above-described exemplary embodiments.

What is claimed is:

1. A machine for cleaning, sanitizing, disinfecting, purifying and/or otherwise beneficially treating surfaces, comprising:
a control system configured to direct, control and vary power and energy output to various components of a machine;
a power unit to supply power to the machine, the power unit connected to the control system;
one or more sources to emit directed energy at one or more targets, the one or more sources connected to the control system; and
a housing to contain the various components of the machine.

2. The machine of claim 1 wherein the one or more sources may include any number and combination of light emitting diodes, lights, lasers, laser diodes, pulsed electromagnetic field emitters and waveguides, cavity magnetrons, magnetic stimulation devices, radio frequency oscillators.

3. The machine of claim 1, further comprising:
an automated movement control unit to automatically control and move the machine; and
one or more location or perimeter sensors to allow the machine to navigate around or over obstacles.

4. The machine of claim 1, further comprising:
a closed loop temperature control unit to monitor and maintain the temperature produced by the machine in or upon the one or more targets being treated.

5. The machine of claim 1, further comprising:
one or more rotatable wheels, tires, rollers, disks, or casters to facilitate movement of the device.

6. The machine of claim 1 whereby the one or more sources are placed on the bottom of the housing unit and directed to a surface below the machine.

7. The machine of claim 1, further comprising:
one or more programmable timer units for keeping track of one or more time parameters, wherein the time parameters may include a length of time the one or more sources are active, a length of time a temperature is maintained, and a length of time directed energy based on an at least one set of parameters is emitted, wherein the at least one set of parameters may include one or more of a wavelength, frequency, amplitude, duration, acceleration, deceleration, pulsation, and pulsation period.

8. A method for treating surfaces, comprising:
selecting an at least one first set of parameters of directed energy, wherein the at least one first set of parameters may include one or more of a wavelength, frequency, amplitude, duration, acceleration, deceleration, pulsation, and pulsation period;
emitting the directed energy based on the selected at least one first set of parameters at a target surface;
measuring in real-time the temperature of the target surface; and
pausing the emitting of directed energy once a trigger occurs, wherein the trigger may be one or more of a passage of a prescribed length of time, a reaching of a threshold temperature, and a maintenance of a temperature for a prescribed length of time.

9. The method of claim 8, further comprising:
selecting an at least one second set of parameters of directed energy, wherein the at least one second set of parameters may include one or more of a wavelength, frequency, amplitude, duration, acceleration, deceleration, pulsation, and pulsation period;
emitting the directed energy based on the selected at least one second set of parameters at the target surface; and
pausing the emitting of directed energy once a trigger occurs, wherein the trigger may be one or more of a passage of a prescribed length of time, a reaching of a threshold temperature, and a maintenance of a temperature for a prescribed length of time.

10. The method of claim 9, further comprising:
selecting an at least one third set of parameters of directed energy, wherein the at least one second set of parameters may include one or more of a wavelength, frequency, amplitude, duration, acceleration, deceleration, pulsation, and pulsation period;
emitting the directed energy based on the selected at least one third set of parameters at the target surface, wherein the at least one third set of parameters may include one or more of a wavelength, frequency, amplitude, duration, acceleration, deceleration, pulsation, and pulsation period; and
pausing the emitting of directed energy once a trigger occurs, wherein the trigger may be one or more of a passage of a prescribed length of time, a reaching of a threshold temperature, and a maintenance of a prescribed temperature for a prescribed length of time.

11. A method for the purification of air, comprising:
selecting an at least one first set of parameters of directed energy, wherein the at least one first set of parameters may include one or more of a wavelength, frequency, amplitude, duration, acceleration, deceleration, pulsation, and pulsation period;
emitting the directed energy, based on the selected at least one first set of parameters, in a chamber from one or more directions;
exposing an unpurified mass of air to the emitted directed energy; and
moving the purified mass of air out of the chamber to allow it to be replaced by an unpurified mass of air.

12. The method of claim 11, further comprising:
adding pre-oxygenated air containing ozone to the purified mass of air.

13. The method of claim 11, further comprising:
moving the mass of air across or through an electrostatic grid.

14. The method of claim 11 further comprising:
pausing the emitting of directed energy once a trigger occurs, wherein the trigger may be one or more of a passage of a prescribed length of time, a reaching of a threshold temperature, and a maintenance of a prescribed temperature for a prescribed length of time.

15. The method of claim 11, further comprising:
selecting an at least one second set of parameters of directed energy, wherein the at least one second set of parameters may include one or more of a wavelength, frequency, amplitude, duration, acceleration, deceleration, pulsation, and pulsation period;
emitting the directed energy based on the at least one second set of parameters in the chamber; and
exposing the mass of air to the emitted directed energy for a prescribed length of time.

16. The method of claim 15 further comprising:
pausing the emitting of directed energy once a trigger occurs, wherein the trigger may be one or more of a passage of a prescribed length of time, a reaching of a threshold temperature, and a maintenance of a prescribed temperature for a prescribed length of time.

17. The method of claim 15, further comprising:
selecting an at least one third set of parameters of directed energy, wherein the at least one third set of parameters may include one or more of a wavelength, frequency, amplitude, duration, acceleration, deceleration, pulsation, and pulsation period; and
emitting the directed energy based on the at least one third set of parameters in the chamber; and
exposing the mass of air to the emitted directed energy for a prescribed length of time.

18. An air vent purification system, comprising:
a chamber to contain and funnel a mass of air;
an at least one air inlet, to allow the movement of air into the chamber;
an at least one air outlet, to allow the movement of air out of the chamber;
one or more sources for emitting directed energy inside the chamber;
an electrostatic grid placed in a portion of the chamber; and
an airflow actuator to facilitate the movement of the mass of air through the chamber.

19. The air vent purification system of claim 18, further comprising:
An at least one inlet for the addition of oxygenated ozone to the purified mass of air.

20. A body part and object sanitation, disinfecting, cleaning, and treatment system, comprising:
one or more outlets for the expulsion of air;
one or more sources for emitting directed energy towards the direction of an at least one target body part or object placed near the one or more outlets;
a sensor that automatically detects the position of the at least one target body part or object;
a signaling mechanism that activates when a trigger occurs, wherein the trigger may be one or more of a passage of a prescribed length of time, a reaching of a threshold temperature, a maintenance of a prescribed temperature for a prescribed length of time, and removal of a target body part or object; and
an automated on-off mechanism that responds to the activated signaling mechanism, and the placement or the removal of the at least one target body part or object near the one or more outlets.

21. A method for sanitization, disinfection, purification or other beneficial treatment of objects, comprising:
placing an at least one target body part or object near one or more outlets for the expulsion of air;
detecting the placement of the at least one target body part or object near the one or more outlets;
activating the expulsion of air;
emitting directed energy at or towards the at least one target body part or object;
detecting a trigger, wherein the trigger may be one or more of a passage of a prescribed length of time, a reaching of a threshold temperature, a maintenance of a prescribed temperature for a prescribed length of time, and removal of a target body part or object; and
based on the detection, halting the expulsion of air and the emitting of directed energy.

22. A device for the sanitization, disinfection, purification and other beneficial treatment of objects, comprising:
a conveyor belt to move an object from one point to another;
one or more points of entry for the objects to be placed onto or into the device;
one or more exits for the objects to be removed from the device;
one or more sources for emitting directed energy;
a closed-loop temperature sensor; and
a housing for the containment of the device in a closed or semi-closed environment.
